(19) United States
(12) Patent Application Publication
(10) Pub. No.: US 2009/0266719 A1
(43) Pub. Date: Oct. 29, 2009

(54) Potentiometric Urea Sensor Based on Ion-Selective Electrode

(76) Inventors: Shen-Kan HSIUNG, Jungli City (TW); Jung-Chuan Chou, Douliou City (TW); Tai-Ping Sun, Jangli City (TW); Chien-Wei Pan, Wandan Township (TW); Nien-Hsuan Chou, Jhongi City (TW)

Correspondence Address: Hung-Chih Tseng;HDLS IPR Services, PO Box 220746, Chantilly, VA, 20153, US

(21) Appl. No.: 12/498,473

(22) Filed: Jul. 7, 2009

Related U.S. Application Data
(63) Continuation-in-part of application No. 10/984,495, filed on Nov. 8, 2004.

(51) Int. Cl. G01N 27/26; C12N 11/14

(52) U.S. Cl. 205/778; 204/403.1; 435/176

(57) ABSTRACT

A sensor for sensing and measuring a concentration of urea in a sample has an ammonium ion selective membrane and urease enzymes immobilized on the ammonium ion selective membrane. The urease enzymes enzymatically convert urea into ammonium ions, which is sensed by said ammonium ion selective membrane and transformed into a signal. A detector system is used for processing signal from said ammonium ion selective membrane to generate a response potential that corresponds to the concentration of urea in the sample.

This application is a continuation in part of the U.S. patent application Ser. No. 10/984,495 filed on Nov. 8, 2004.

### BACKGROUND OF THE INVENTION

1. Field of the Invention

The present invention relates to a sensor device, and more particularly to a potentiometric urea sensor for sensing and measuring a concentration of ammonium ions in a liquid sample.

2. Description of the Prior Art

In recent years, with the rapid progress in electronic technologies, the technology of bio-device has been further improved and applied to design better sensors for determining urea in biological samples such as blood, blood components and urine. In clinical examinations such as in hospital/clinic, blood urea nitrogen (BUN) assay useful in assessing the kidney function. A typical BUN value of a healthy human being is in a range of about 8-20 mg/dl. A BUN value higher than 20 mg/dl indicates impaired renal function, congestive heart failure, acute myocardial infarction, dehydration or excessive protein intake. On the other hand, a BUN value lower than 8 mg/dl indicates liver failure, malnutrition, anabolic steroid use or siliac disease. Thus, it is highly desirable to measure the concentrations of biological substances for evaluating/monitoring the function of various organs of the human body. Various prior arts propose indirect measurement of the concentration of urea such as measuring pH value or volume of ammonia gas transformed by ammonium ions, spectral analysis or enzyme method. However, so far, none of the prior arts present any method of directly sensing and measuring the concentration of the ammonium ions. Accordingly, a device and a method for directly sensing and measuring ammonium ions are highly desirable.

### SUMMARY OF THE INVENTION

The present invention is directed to a potentiometric urea sensor for sensing and measuring a concentration of ammonium ions.

The present invention is directed to a potentiometric urea sensor comprising an ammonium ion-selective membrane for sensing and measuring a concentration of ammonium ions in a biological sample.

The present invention is also directed to a method for fabricating a potentiometric urea sensor for sensing and measuring a concentration of ammonium ions in a biological sample.

In an embodiment, the potentiometric urea sensor can be easily fabricated at a lower cost and protected from heat and light.

In one embodiment, the potentiometric urea sensor for sensing and measuring the concentration of urea in a biological sample comprises an ammonium ion selective membrane and urease enzymes immobilized on at least a portion or over the ammonium ion selective membrane, and a detector system for processing signals from the sensor.

In one embodiment, the ammonium ion selective membrane comprises a water-permeable matrix on the ammonium ion selective membrane immobilizing the urease enzymes.

In one embodiment, the ammonium ion selective membrane comprises a plasticized polyvinyl chloride and nonactin.

In another embodiment, the urease enzymes may be immobilized on another layer and then disposed over and in contact with at least a portion of the ammonium ion selective membrane. The ammonium ion selective membrane comprises plasticized polyvinylchloride and nonactin, and the urease enzyme is immobilized on a water-permeable matrix.

In an embodiment, the potentiometric urea sensor comprises a substrate, a non-insulating solid-state ion sensing electrode, a sensing window, a conductive line, an ammonium ion selective membrane, an enzyme layer and a read-out circuit. The non-insulating solid-state ion sensing membrane is formed on the carbon based substrate and is adopted for sensing a pH value of a solution. The conductive line is disposed on the carbon based substrate and serves as a transmission line for the sensing signal. The carbon based substrate is connected to the single sensing window or array sensing windows structure on the carbon based substrate. Compare to the single sensing structure, the array sensors have the following advantages: (1) Multiple measurement to save the usage of sample, (2) Increasing the signal to noise ratio (S/N) to improved the design of output interface, (3) Increasing the usage lifetime of sensor to have good commercial value. The ammonium ion-selective membrane is disposed in the sensing window and the enzyme layer is immobilized in the sensing window. The readout circuit is connected to the conductive line for reading the sensing signal from the ammonium ion-selective electrode.

### BRIEF DESCRIPTION OF THE DRAWINGS

FIGS. 1A-1C illustrates a schematic process for fabricating a potentiometric urea sensor according to an embodiment of the present invention.

FIG. 2A illustrates top and cross sectional views of the potentiometric urea sensor according to an embodiment of the present invention;

FIG. 2B illustrates top view of an array of potentiometric urea sensors according to an embodiment of the present invention;

FIG. 3 illustrates a view of a detector system for a potentiometric urea sensor according to an embodiment of the present invention;

FIG. 4 is a linear calibration curve of response potentials measured by a potentiometric urea sensor using solutions of known concentrations of urea ranging from 0.1 mmole/l to 1 mole/l;

FIG. 5 illustrates a relationship curve between the response potentials and time of the potentiometric urea sensor;

FIG. 6 illustrates a linear calibration curve of a response potential measured by a potentiometric urea sensor;

FIG. 7 is a calibration curve of response potentials measured by a potentiometric urea sensor using solutions of known concentrations of urea ranging from 0.8 .mu.mole/l to 10 mmole/l at pH=7.5;

FIG. 8 is a calibration curve of a response potential measured by a potentiometric urea sensor at different pH values; and

FIG. 9 illustrates the max response potential measured by a potentiometric urea sensor at different pH values.

### DETAILED DESCRIPTION OF THE PREFERRED EMBODIMENT

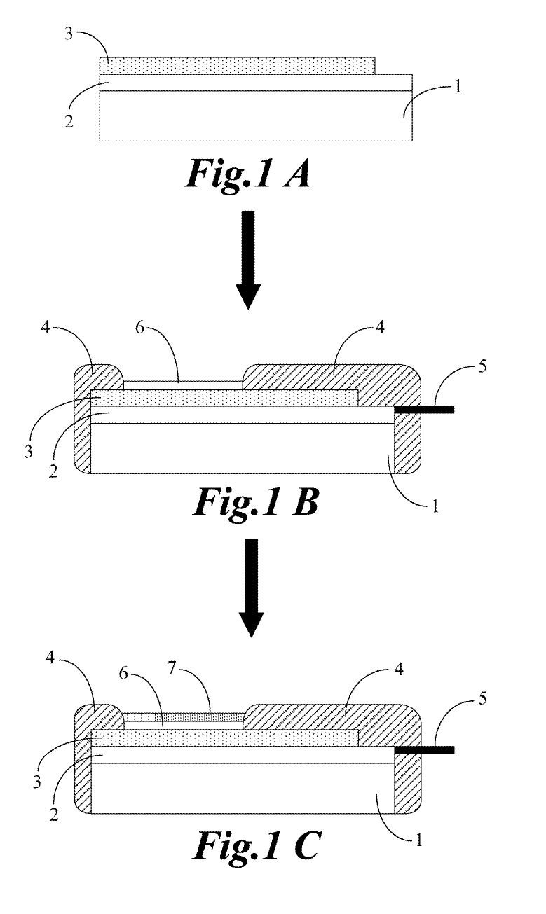

Referring to FIG. 1A, the potentiometric urea sensor comprising an ammonium ion-selective membrane may be fabricated as follows. First, a substrate 1 is provided. The substrate 1 may be comprised of a ceramic, glass, or the like. Next, a carbon-based film 2 is disposed over the substrate 1. The carbon-based film may be comprised of carbide and serves as an electrode (we can also add other examples here). Next, a SnO₂ film 3 is sputtered over the carbon-based layer 2 and a conductive line 5 is fixed on said carbon-based film 2. Next, a tin dioxide SnO₂ film 3 with a thickness about 2000 angstrom is sputtered on the substrate 1.

Next, as illustrated in FIG. 1B, the conductive line 5 is fixed at an end portion of the SnO₂ film 2, and is exposed by the SnO₂ film 3. The conductive line 5 may be comprised of a silver paste. The conductive line 5 serves as a transmission line for the sensing signal and is encapsulated by an epoxy resin 4. The encapsulation 4 defines a sensing window with an area of about 2×2 mm². Thus, the fabrication of the solid-state pH ion sensing electrode is completed. Next, an ammonium ion-selective membrane 6 is disposed in the sensing window. The ammonium ion-selective membrane 6 may be formed by using a solution including: (a1) poly(vinyl chloride) carboxylated, sebacate, DOS: 66% and ammonium ion-selective substance (Nonactin): 1%; and (a2) a conjugate base (Tris(hydroxymethyl)-aminomethane, Tris): 20 mmole/l and a conjugate acid (Ethylen—diaminetetraacetic acid (disodium salt), EDTA): 1.0 mmole/l, the pH value is adjusted to be 7.5 by hydrochloric acid(HCl).

Other plasticizers suitable for use may include, but are not limited to tris(2-ethylhexyl)phosphate, nitrocymene, 2-nitrophenyloctyl ether, dibutyl sebacate, diethyl adipate, phthalates, propylene carbonate, 5-phenylpentanol, or mixtures thereof. Still other binders and ionophore combinations may occur to those skilled in the art, which are within the scope of the present invention.

Next, as illustrated in FIG. 1C, urease enzymes 7 are immobilized in the sensing window on the ammonium ion-selective membrane 6. The urease enzymes 7 may be immobilized using a photopolymer including, for example, a poly(vinyl alcohol)-styrylpyridinium (PVA-SbQ) including Poly(vinylalcohol) having Styrylpyridinium Groups, (degree of polymerization 3500, degree of saponification 88, betaine Sbq 1.05 mol %, solid content 10.22 mol %, pH 5.7, SPP-H-13). The composition of the urease enzyme film 7 in a 125 mg/100 ml, pH=7.0 includes 5 mmole/l phosphate solution, PVA-SbQ mixed with a urea solution (a 10 mg/100 μl, pH 7.0, 5 mmole/l phosphate solution) in a ratio of 1:1.

Hereinafter, the process of immobilization of urease enzymes 7 may be described as follows. First, the solution of urea/PVA-SbQ about 10 μl may be dropped on the ammonium ion selective membrane 6, and then the ammonium ion selective membrane 6 may be irradiated with a 4 W ultraviolet light at wavelength 365 nm for 20 minutes to polymerize the photopolymer and thereby immobilize the urease enzymes on the ammonium ion-selective membrane 6 in the sensing window to complete the fabrication of the potentiometric urea sensor device.

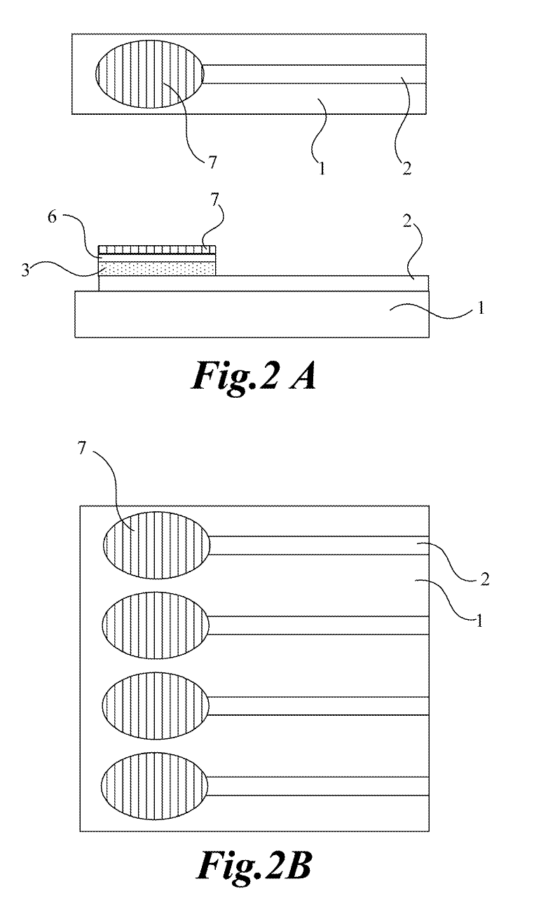

FIG. 2A-B illustrates top and cross sectional views of the potentiometric urea sensor; and a top view of an array of potentiometric urea sensors according to an embodiment of the present invention.

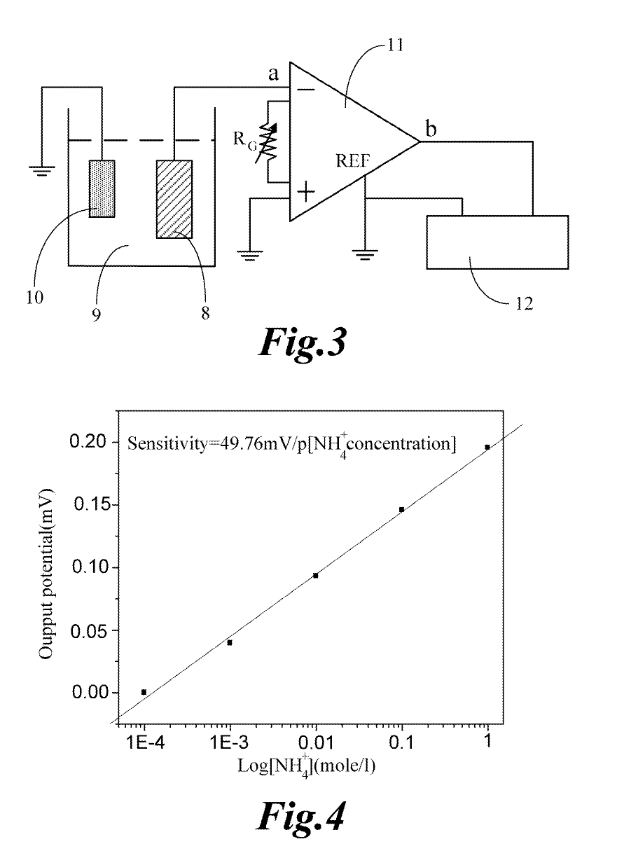

As illustrated in FIG. 3, a detector system for a urea sensor comprises a readout circuit including an amplifier 11. The urea sensor 8 placed in a buffer solution 9 for measuring urea is connected to the negative input a of the instrumentation amplifier, while a silver/chloride silver electrode 10 correspondingly provides a stable reference potential, so as to measure the response potential of the sensor. The output end b of the instrumentation amplifier 11 is connected to a multi-function digital meter 12.

The operation of the urea sensor may be described as follows. At step 1, an amplifier is used as the readout circuit of the potentiometric urea sensor device. At step 2, the potentiometric urea sensor device is placed into a buffer solution for some time until a stable response potential is read, which is taken as the reference potential. At step 3, the potentiometric urea sensor is placed into a sample solution, for example a blood sample. In the blood sample, the urea is first enzymatically converted to NH₄⁺ and HCO₃⁻ by the immobilized urease enzymes on the ammonium ion selective membrane. Next, the NH₄⁺ may be directly sensed by the ammonium ion-selective membrane 6 and which in turn is read as a signal by the read-out circuit to generate a response potential, whose value corresponds to the concentration of the ammonium ions. The measured concentration of ammonium ions provides an estimated concentration of the urea in blood.

FIG. 4 is a linear calibration curve of a response potential, measured by an ammonium ion-selective electrode using the ammonium concentration ranging from 0.1 mmole/l to 1 mole/l, using the measurement circuit illustrated in FIG. 3. The sensitive characteristic of the ammonium ion-selective electrode is measured using the ammonium concentration ranging from 0.1 mmole/l to 1 mole/l, and via the calculation of the linear calibration curve of the response potential. This clearly indicates that the measurement of the ammonium ion concentration is both precise as well as reliable within the concentration range 0.1-1.0 mmole/l.

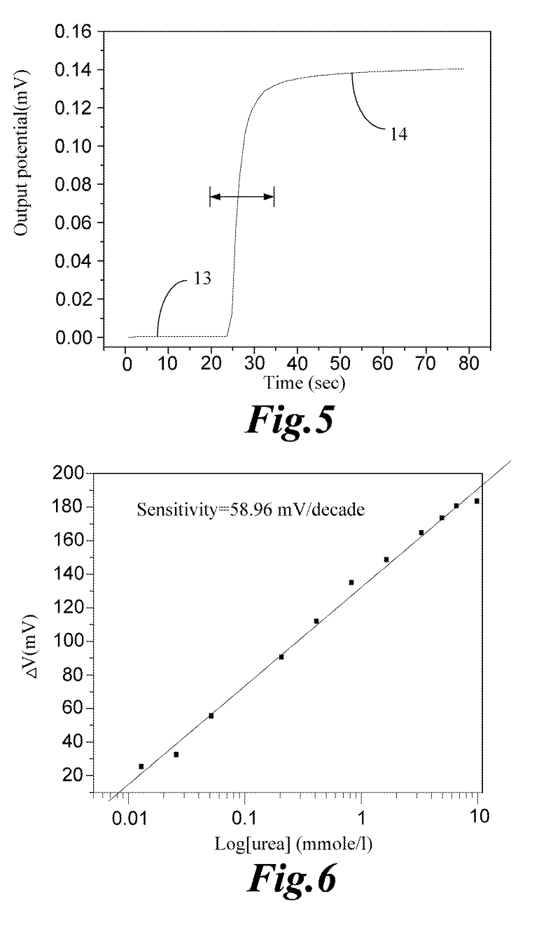

FIG. 5 illustrates a relationship curve between the response potential and time of the potentiometric urea sensor device using the measurement circuit illustrated in FIG. 3. First, the potentiometric urea sensor device is placed into a Tris-HCL buffer solution (20 mmole/l, pH 7.5). After the potential 13 is stabilized, the potentiometric urea sensor device is used to measure the response potential 14 of a solution for measuring enzyme. As can be seen from the curve, the response potential can reach up to 90% of the max response potential even when the response time less than 15 sec. (about 20 sec. to about 35 sec.).

FIG. 6 illustrates a linear calibration curve of the response potentials measured by the potentiometric urea sensor with a linear concentration range from 0.02 to 1.0 mmole/l, using the detector system illustrated in FIG. 2. After calculation with the chart, the sensitivity of the potentiometric urea sensor is obtained.

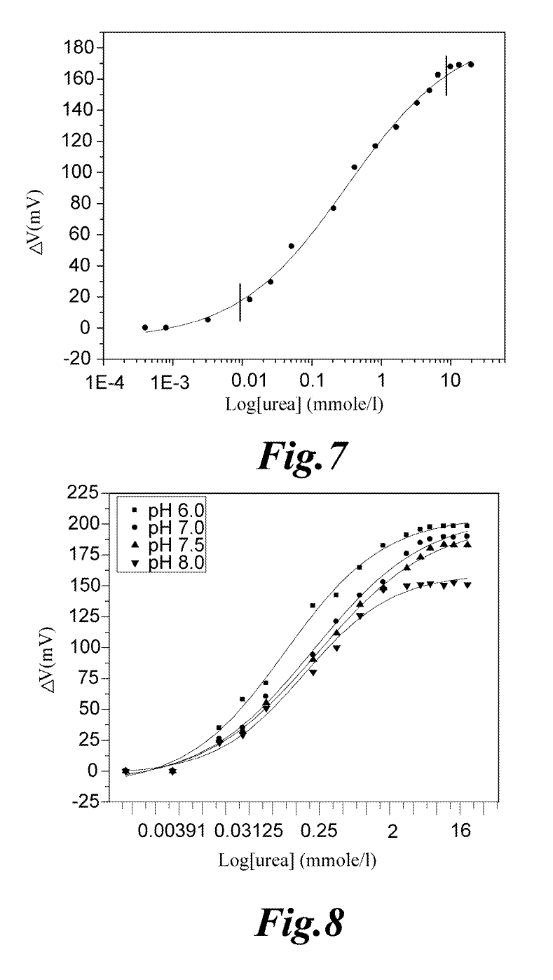

As illustrated in FIG. 7, the response potential results of a solution for measuring urea with a concentration ranging from 0.8 .mu.mole/l to 10 mmole/l and the pH value 7.5, are measured by the potentiometric urea sensor, using the measurement circuit illustrated in FIG. 2. As can be seen from the curve, the linear measurement range of the urea sensing device is from 0.02 mmole/l to 10 mmole/l, minimum measurement is 3 .mu.mole/l. Thus, the linear measurement range can be within the standard urea concentration range of a human blood (2.8 mmole/l to 7.12 mmole/l).

As illustrated in FIG. 8, the response potentials of a urea sample with a concentration ranging from 0.8 .mu.mole/l to 10 mmole/l at different pH values, are measured by the potentiometric urea sensor device using the measurement circuit illustrated in FIG. 3. The object is to observe how the pH value variation of the solution to be measured may affect the response potential and the calibration curve. As illustrated in FIG. 7, the higher pH value of the solution, the narrower linear measurement range and the smaller response potential difference. Thus, the urease enzyme activity was significantly reduced at pH values above pH 7.4. Urease enzyme activity at pH 8.0 is typically about 50% that of pH 7.0. Secondly, a greater sensor response at high [BUN] is observed with the added buffer. The reasons for the sensor response improvement are discussed below.

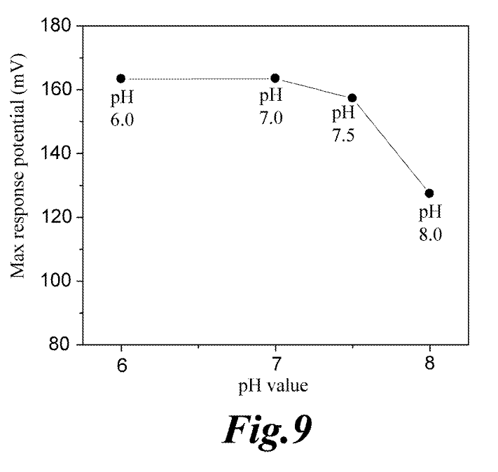

FIG. 9 is a chart of the max response potentials obtained from the measured results in FIG. 8 using solutions to be measured with different pH values and Table 1 lists the values of max response potentials and the linear measurement ranges. As the measured results illustrated in FIG. 8 and FIG. 9, the potentiometric urea sensor has more stable response potential and measurement range when pH ranges from 6 to 7.5. Considering the conditions, such as sensitivity of the ammonium ion-selective electrode within a pH range from 6.0 to 8.0 overlapping with the human blood pH range 7.35-7.45, the potentiometric urea sensor is suitable for measuring the urea concentration in blood.

TABLE 1
the Measured Results Obtained From Measured Environments
With Different pH, Using the Potentiometric Urea Sensor

| pH value of the measured environment | pH 6.0 | pH 7.0 | pH 8.0 |
|---|---|---|---|
| max response potential (mV) | 198.067 | 189.78 | 151.09 |
| linear measurement range (mmole/l) | 0.4-10 | 0.4-6.5 | 0.4-5 |

Accordingly compared to prior arts, the present invention has at least the following advantages. The urease enzymes are immobilized via a chemical cross-linking reagent or physical adsorption. The urease enzymes convert the urea into ammonium ions whose concentration is then directly measured as response potential which corresponds to the concentration of urea in the sample. Thus, the measurement of the urea concentration is not only rapid but also more accurate compared to the prior arts. Besides, the potentiometric urea sensor of the present invention can be fabricated by using a simpler and standard semiconductor process and therefore the fabrication cost is reduced and the through-put is increased.

The above description is given by way of example, and not limitation. Given the above disclosure, one skilled in the art could devise variations that are within the scope and spirit of the invention disclosed herein, including configurations ways of the recessed portions and materials and/or designs of the attaching structures. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. A detector system, comprising:
a sensor, including an ammonium ion selective membrane, for sensing and measuring a concentration of urea in a sample; and
urease enzymes, immobilized over and in contact with at least a portion of said ammonium ion selective membrane, for enzymatically converting urea into ammonium ions, which is sensed by said ammonium ion selective membrane and transformed into a signal to generate a response potential corresponding to the concentration of urea in the sample.

2. The device of claim 1, wherein said ammonium ion selective membrane includes an ammonium ionophore.

3. The device of claim 1, wherein said ammonium ion selective membrane includes nonactin.

4. The device of claim 1, wherein said ammonium ion selective membrane includes plasticized polymer.

5. The device of claim 1, in which said ammonium ion selective membrane comprises a potentiometric ammonium ion selective electrode.

6. The device of claim 1, wherein said enzymes comprise urease enzymes.

7. The device of claim 1, wherein said enzymes are immobilized in a layer comprising a water-permeable matrix.

8. The device of claim 1, wherein said enzymes are immobilized via a chemical cross-linking reagent.

9. The device of claim 1, wherein a photopolymer is utilized to immobilize the urea enzyme film on said ammonium ion-selective membrane.

10. The device of claim 1, wherein said enzymes are immobilized via physical absorption.

11. The device of claim 1, wherein an array of potentiometric urea sensors are provided, each potentiometric urea sensor including a carbon based substrate being connected to a single sensing window or array sensing windows structure on the carbon based substrate.

12. The device of claim 1, wherein said ammonium ion-selective membrane comprises: Poly(vinyl chloride) carboxylated: 33%, dimethyl sebacate: 66%, ammonium ion-selective substance: 1%; conjugate base: 20 mmole/l and conjugate acid: 1.0 mmole/l, the pH value is adjusted to be 7.5 by hydrochloric acid(HCl).

13. A method for assaying urea in a sample, comprising:
contacting a sample containing urea with a sensor having an ammonium ion-selective membrane and urease enzymes immobilized thereon;
enzymatically converting said urea into ammonium ions by said urease enzymes; and
measuring a concentration of said ammonium ions using a detector system.

14. The method of claim 13, wherein said concentration of ammonium ion measured is a function of a concentration of urea in sample.

15. The method of claim 13, wherein said sensor is potentiometric and function substantially logarithmic.

16. The method of claim 13, wherein said sensor is calibrated by exposing said sensor to an aqueous fluid, which contains a known amount of ammonium ion, before or after said sample is contacted with the sensor.

17. A method for fabricating a urea sensor, comprising:
providing a detector system comprising a sensor;
forming an ammonium ion selective membrane over said sensor; and
immobilizing urease enzymes on said ammonium ion selective membrane.

18. The method of claim 17, wherein said ammonium ion selective membrane includes an ammonium ionophore.

19. The method of claim 17, wherein said enzymes are immobilized in a layer comprising a water-permeable matrix.
